(19) United States
(12) Patent Application Publication
(10) Pub. No.: US 2014/0242259 A1
(43) Pub. Date: Aug. 28, 2014

(54) Method for Smoothing Cannula Bevel Heel

(71) Applicant: Christopher S. Young, Lakewood Ranch, FL (US)

(72) Inventor: Christopher S. Young, Lakewood Ranch, FL (US)

(21) Appl. No.: 14/172,102

(22) Filed: Feb. 4, 2014

Related U.S. Application Data
(60) Provisional application No. 61/767,973, filed on Feb. 22, 2013.

(51) Int. Cl. A61M 5/32

(52) U.S. Cl. CPC A61M 5/3286
USPC 427/2.28

(57) ABSTRACT

A method for smoothing sharp areas on the bevel heel of a cannula comprises initially grinding a cannula end portion to form a bevel with a distal tip and an axially opposite heel. An ultraviolet-curable adhesive is applied to the heel. The heel is then exposed to ultraviolet radiation and is ultimately cured. It will be appreciated that the smoothing is accomplished without blasting glass beads at the cannula.

### BACKGROUND

This disclosure relates generally to a cannula and to methods for mass manufacturing cannulas. More particularly, this disclosure relates to methods wherein the cannulas are sharpened by grinding a bevel point proximate the tip.

Medical units employing cannulas are typically manufactured by multiply inserting a cannula into a tube. A series of tube/cannula workpieces is then supplied to a work station wherein the distal ends of each needle is ground to form a bevel to provide a sharpened point. Each cannula, of course, is a thin steel elongated member which includes an axial passageway from the tube to the bevel. The heel portion of the bevel, sometimes referred to as an anti-core heel, is typically, somewhat roughened as a result of grinding.

For certain cannulas it is highly desirable that the heel portion of the bevel be smoothed or otherwise treated to reduce the roughened area. This is particularly true for Huber needles. Anti-core heels on the needle bevels conventionally are smoothed by blasting glass beads after the needle points are ground. While such techniques are generally effective, there are certain applications in which a dull bevel heel is required. There are limits to blasting glass beads in a more intense bombardment because of the potential to damage other parts of the bevel and the practical structural limit to the effectiveness of the smoothing process.

### SUMMARY

Briefly stated, a method for dulling or smoothing sharp areas on the bevel heel of a cannula comprises grinding the cannula end portion to form a bevel with a distal tip and an axially opposite heel. The method further comprises applying an ultraviolet curable adhesive to the heel, exposing the adhesive to ultraviolet radiation and allowing the adhesive to cure.

Preferably, a medical grade adhesive is employed. The step of applying adhesive is accomplished by a dispensing station and dispensing the adhesive from a dispenser having an output delivery tube with a very small diameter. In one embodiment, the method comprises positioning the heel under an ultraviolet lamp and directing ultraviolet radiation at the heel after adhesive has been applied. The method in one preferred embodiment also comprises exposing the bevel to ultraviolet radiation for a duration of three seconds or less.

### BRIEF DESCRIPTION OF THE DRAWINGS

FIG. 1 is a perspective view of a portion of a cannula needle after an initial grinding step;

FIG. 2 is a top plan view of a cannula portion after a subsequent manufacturing step;

FIG. 3 is a side sectional view of the cannula portion of FIG. 2 taken along the line A-A thereof; and

FIG. 4 is a schematic view, illustrating a dispenser and an end portion of a cannula, partially shown in phantom, further illustrating a step in a method for smoothing the heel.

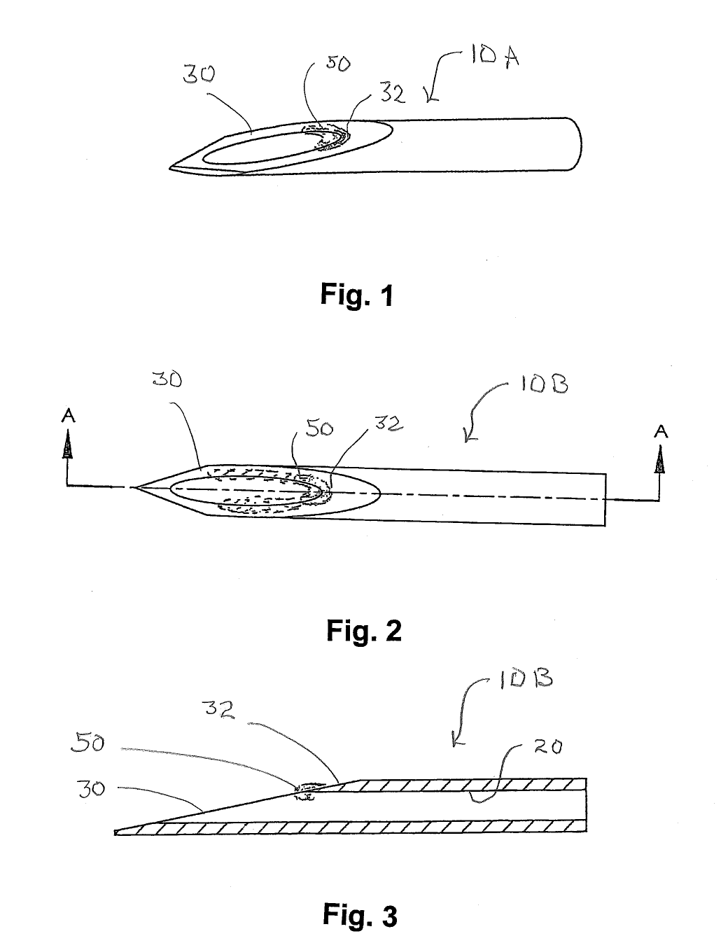

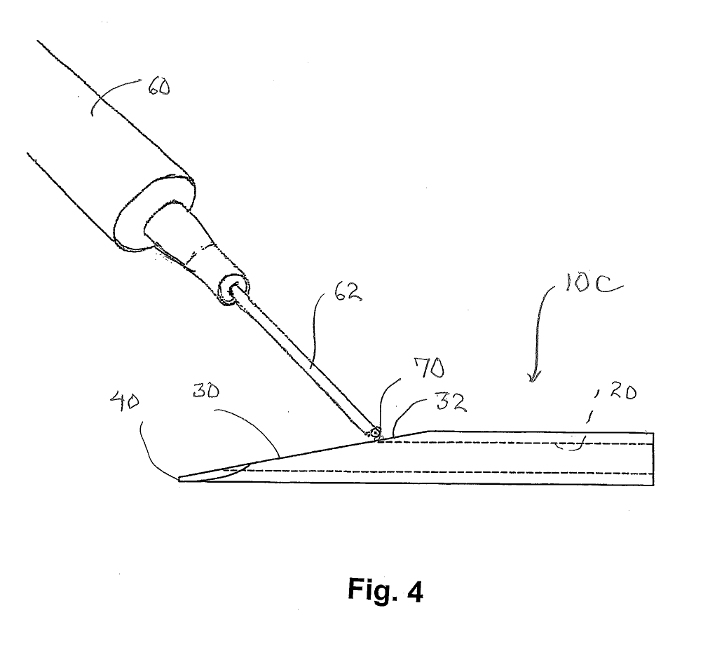

### DETAILED DESCRIPTION

With reference to the drawings, wherein like numerals represent like parts throughout the Figures, a cannula workpiece in various manufacturing stages is generally designated by the numeral 10 together with the letter indicating the various stages.

The cannula 10 is a thin elongated metal member formed of stainless steel and has an axial passageway 20. Typically the cannula is a relatively thin member such as a 30 gauge needle. After the cannula has been cut to the proper length, the cannula is sharpened by grinding one end. The grinding forms a bevel 30 through which the passageway 20 opens and a sharpened distal point 40. The bevel area adjacent the passageway is sometimes referred to as the anti-core heel 32.

The cannulas are typically disposed and oriented in a side by side disposition during the manufacturing process. After the bevel has been ground to form the sharpened point, typically a relatively roughened area 50 is formed at the anti-core heel, closely adjacent the passageway or core.

A dispensing unit 60, which has a small diameter dispensing tip 62, is positioned in the proximity of the heel area to deliver and provide a very precise amount of very low viscosity adhesive 70. In one embodiment, the adhesive 70 is also applied along the sides of the bevel. Alternatively, multiple cannulas are serially positioned adjacent the dispensive unit 60. The preferred adhesive is an adhesive which cures on exposure to ultraviolet (UV) radiation.

After the adhesive is applied 70, the cannula, especially in the region of the bevel heel 32, is exposed to ultraviolet radiation via a UV lamp or other means. Each of the bevel heels 32 may be serially positioned at a curing station, such as under a UV light. Once the adhesive is cured, the heel portion of the bevel no longer exhibits a roughened, sharpened area and has a dull or smooth surface configuration.

Preferably the adhesive 70 is a medical grade adhesive, such as Dymax™ 1180-M UV cure adhesive or equivalent. The exposure to the ultraviolet radiation is preferably limited three seconds or less although other curing times may be employed.

It will be appreciated that the dulled or smoothed areas adjacent the anti-core heel 32 are achieved without excessive blasting of glass beads against the beveled end of the needle. The smoothing manufacturing step is accomplished in a highly efficient manner without jeopardizing the integrity of the finished cannula and can be accomplished on a high volume manufacturing scale.

1. A method for smoothing sharp portions on the bevel heel of a cannula comprising:
grinding a cannula end portion to form a bevel with a distal tip and an axially opposite heel;
applying an ultraviolet curable adhesive to the heel;
exposing the adhesive to ultraviolet radiation; and
allowing the adhesive to cure.

2. The method of claim 1 wherein the adhesive is a medical grade adhesive.

3. The method of claim 1 wherein the step of applying adhesive is accomplished by a dispensing station comprising a dispenser having an output delivery tube with a very small diameter and dispensing the adhesive from the output delivery tube.

4. The method of claim 1 further comprising positioning the heel under an ultraviolet lamp.

5. The method of claim 1 further comprising directing ultraviolet radiation at the heel after adhesive has been applied.

6. The method of claim 1 wherein the step of exposing the adhesive to ultraviolet radiation has a duration of 3 seconds or less.

7. A method for smoothing sharp portions on the bevel heels of a multiplicity of cannulas comprising:
grinding end portions of each cannula to form a bevel with a distal tip and an axially opposite heel;
applying an ultraviolet curable adhesive to the heels of the cannulas; and
exposing the adhesive to ultraviolet radiation.

8. The method of claim 7 wherein the adhesive is a medical grade adhesive.

9. The method of claim 7 wherein the step of applying adhesive is accomplished by a dispensing station comprising a dispenser having an output delivery tube with a very small diameter and dispensing the adhesive from the output delivery tube.

10. The method of claim 7 further comprising positioning the heels under an ultraviolet lamp.

11. The method of claim 7 further comprising directing ultraviolet radiation at the heels after adhesive has been applied.

12. The method of claim 7 wherein the step of exposing the adhesive to ultraviolet radiation has a duration of 3 seconds or less for each cannula.

13. A method for smoothing sharp portions on the bevel heel of a cannula comprising:
grinding the cannula end portion to form a bevel with a distal tip and an axially opposite heel;
applying a curable adhesive to the heel; and
curing the adhesive to form a smooth bevel heel.

14. The method of claim 13 wherein the step of applying adhesive is accomplished by a dispensing station comprising a dispenser having an output delivery tube with a very small diameter and dispensing the adhesive from the output delivery tube.
